SEQUENCE LISTING (79 sequences)

SEQ ID NO: 1 - DNA, 1500 bp, Homo sapiens
tctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtg

SEQ ID NO: 2 - DNA, 1500 bp, Homo sapiens
atctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaagg

SEQ ID NO: 3 - DNA, 27 bp, Homo sapiens
ttgaccaatagtcttagagtatccagt

SEQ ID NO: 4 - DNA, 27 bp, Artificial Sequence
  Mutant_01
ttgaccaatagtgatagagtatccagt

SEQ ID NO: 5 - DNA, 27 bp, Artificial Sequence
  Mutant_02
ttgaccaatagagatagagtatccagt

SEQ ID NO: 6 - DNA, 26 bp, Artificial Sequence
  Mutant_03
ttgaccaataagatagagtatccagt

SEQ ID NO: 7 - DNA, 26 bp, Artificial Sequence
  Mutant_04
ttgaccaatatgatagagtatccagt

SEQ ID NO: 8 - DNA, 32 bp, Homo sapiens
cagccttgccttgaccaatagccttgacaagg

SEQ ID NO: 9 - DNA, 32 bp, Artificial Sequence
  Mutant_05
cagccttgccttgacagatagccttgacaagg

SEQ ID NO: 10 - DNA, 32 bp, Artificial Sequence
  Mutant_06
cagccttgccttgactgatagccttgacaagg

SEQ ID NO: 11 - DNA, 32 bp, Artificial Sequence
  Mutant_07
cagccttgccttgagagatagccttgacaagg

SEQ ID NO: 12 - DNA, 32 bp, Artificial Sequence
  Mutant_08
cagccttgccttgagtgatagccttgacaagg

SEQ ID NO: 13 - DNA, 32 bp, Artificial Sequence
  Mutant_09
cagccttgccttgaaagatagccttgacaagg

SEQ ID NO: 14 - DNA, 32 bp, Artificial Sequence
  Mutant_10
cagccttgccttgaatgatagccttgacaagg

SEQ ID NO: 15 - DNA, 32 bp, Artificial Sequence
  Mutant_11
cagccttgccttgatagatagccttgacaagg

SEQ ID NO: 16 - DNA, 32 bp, Artificial Sequence
  Mutant_12
cagccttgccttgattgatagccttgacaagg

SEQ ID NO: 17 - DNA, 32 bp, Artificial Sequence
  Mutant_13
cagccttgccttgataaatagccttgacaagg

SEQ ID NO: 18 - DNA, 33 bp, Artificial Sequence
  Mutant_14
cagccttgccttgataaaatagccttgacaagg

SEQ ID NO: 19 - DNA, 31 bp, Artificial Sequence
  Mutant_15
cagccttgccttgataatagccttgacaagg

SEQ ID NO: 20 - DNA, 28 bp, Artificial Sequence
  Mutant_16
cagccttgccttgatagccttgacaagg

SEQ ID NO: 21 - DNA, 19 bp, Artificial Sequence
  Mutant_17
cagccttgccttgataagg

SEQ ID NO: 22 - DNA, 20 bp, Artificial Sequence
  Mutant_18
cagccttgccttgataaagg

SEQ ID NO: 23 - DNA, 23 bp, Homo sapiens
tatctgtctgaaacggtccctgg

SEQ ID NO: 24 - DNA, 29 bp, Artificial Sequence
  Mutant_19
tatctgtctgaaacggtagataaccctgg

SEQ ID NO: 25 - DNA, 33 bp, Homo sapiens
cactatctcaatgcaaatatctgtctgaaacgg

SEQ ID NO: 26 - DNA, 25 bp, Artificial Sequence
  Mutant_20
cactatctcaatgcaacagaaacgg

SEQ ID NO: 27 - DNA, 23 bp, Homo sapiens
tccctgaacttttcaaaaattgg

SEQ ID NO: 28 - DNA, 24 bp, Artificial Sequence
  Mutant_21
tccctgaacttttcagataattgg

SEQ ID NO: 29 - DNA, 25 bp, Artificial Sequence
  Mutant_22
tccctgaacttttcagataaattgg

SEQ ID NO: 30 - DNA, 26 bp, Artificial Sequence
  Mutant_23
tccctgaacttttcagataaaattgg

SEQ ID NO: 31 - DNA, 26 bp, Artificial Sequence
  Mutant_24
tccctgaacttttcagatagaattgg

SEQ ID NO: 32 - DNA, 23 bp, Homo sapiens
actggatactctaagactattgg

SEQ ID NO: 33 - DNA, 23 bp, Homo sapiens
cttgtcaaggctattggtcaagg

SEQ ID NO: 34 - DNA, 23 bp, Homo sapiens
gtttgccttgtcaaggctattgg

SEQ ID NO: 35 - DNA, 23 bp, Homo sapiens
tggtcaagtttgccttgtcaagg

SEQ ID NO: 36 - DNA, 23 bp, Homo sapiens
cttgaccaatagccttgacaagg

SEQ ID NO: 37 - DNA, 23 bp, Homo sapiens
tatctgtctgaaacggtccctgg

SEQ ID NO: 38 - DNA, 23 bp, Homo sapiens
atgcaaatatctgtctgaaacgg

SEQ ID NO: 39 - DNA, 23 bp, Homo sapiens
tccctgaacttttcaaaaattgg

SEQ ID NO: 40 - DNA, 84 bp, Artificial Sequence
  ssODN_1
cctagccagccgccggcccctggcctcactggatactctatcactattggtcaagtttgccttgtcaaggctattggtcaaggc

SEQ ID NO: 41 - DNA, 84 bp, Artificial Sequence
  ssODN_2
cctagccagccgccggcccctggcctcactggatactctatctctattggtcaagtttgccttgtcaaggctattggtcaaggc

SEQ ID NO: 42 - DNA, 83 bp, Artificial Sequence
  ssODN_3
cctagccagccgccggcccctggcctcactggatactctatcttattggtcaagtttgccttgtcaaggctattggtcaaggc

SEQ ID NO: 43 - DNA, 83 bp, Artificial Sequence
  ssODN_4
cctagccagccgccggcccctggcctcactggatactctatcatattggtcaagtttgccttgtcaaggctattggtcaaggc

SEQ ID NO: 44 - DNA, 82 bp, Artificial Sequence
  ssODN_5
ctctaagactattggtcaagtttgccttgtcaaggctatctgtcaaggcaaggctggccaacccatgggtggagtttagcca

SEQ ID NO: 45 - DNA, 82 bp, Artificial Sequence
  ssODN_6
ctctaagactattggtcaagtttgccttgtcaaggctatcagtcaaggcaaggctggccaacccatgggtggagtttagcca

SEQ ID NO: 46 - DNA, 82 bp, Artificial Sequence
  ssODN_7
ctctaagactattggtcaagtttgccttgtcaaggctatctctcaaggcaaggctggccaacccatgggtggagtttagcca

SEQ ID NO: 47 - DNA, 82 bp, Artificial Sequence
  ssODN_8
ctctaagactattggtcaagtttgccttgtcaaggctatcactcaaggcaaggctggccaacccatgggtggagtttagcca

SEQ ID NO: 48 - DNA, 82 bp, Artificial Sequence
  ssODN_9
ctctaagactattggtcaagtttgccttgtcaaggctatctttcaaggcaaggctggccaacccatgggtggagtttagcca

SEQ ID NO: 49 - DNA, 82 bp, Artificial Sequence
  ssODN_10
ctctaagactattggtcaagtttgccttgtcaaggctatcattcaaggcaaggctggccaacccatgggtggagtttagcca

SEQ ID NO: 50 - DNA, 82 bp, Artificial Sequence
  ssODN_11
ctctaagactattggtcaagtttgccttgtcaaggctatctatcaaggcaaggctggccaacccatgggtggagtttagcca

SEQ ID NO: 51 - DNA, 82 bp, Artificial Sequence
  ssODN_12
ctctaagactattggtcaagtttgccttgtcaaggctatcaatcaaggcaaggctggccaacccatgggtggagtttagcca

SEQ ID NO: 52 - DNA, 82 bp, Artificial Sequence
  ssODN_13
ctctaagactattggtcaagtttgccttgtcaaggctatttatcaaggcaaggctggccaacccatgggtggagtttagcca

SEQ ID NO: 53 - DNA, 83 bp, Artificial Sequence
  ssODN_14
ctctaagactattggtcaagtttgccttgtcaaggctattttatcaaggcaaggctggccaacccatgggtggagtttagcca

SEQ ID NO: 54 - DNA, 81 bp, Artificial Sequence
  ssODN_15
ctctaagactattggtcaagtttgccttgtcaaggctattatcaaggcaaggctggccaacccatgggtggagtttagcca

SEQ ID NO: 55 - DNA, 78 bp, Artificial Sequence
  ssODN_16
ctctaagactattggtcaagtttgccttgtcaaggctatcaaggcaaggctggccaacccatgggtggagtttagcca

SEQ ID NO: 56 - DNA, 81 bp, Artificial Sequence
  ssODN_17
cctcactggatactctaagactattggtcaagtttgccttatcaaggcaaggctggccaacccatgggtggagtttagcca

SEQ ID NO: 57 - DNA, 82 bp, Artificial Sequence
  ssODN_18
cctcactggatactctaagactattggtcaagtttgcctttatcaaggcaaggctggccaacccatgggtggagtttagcca

SEQ ID NO: 58 - DNA, 81 bp, Artificial Sequence
  ssODN_19
tggctaaactccacccatgggttggccagccttgccttgataaggcaaacttgaccaatagtcttagagtatccagtgagg

SEQ ID NO: 59 - DNA, 82 bp, Artificial Sequence
  ssODN_20
tggctaaactccacccatgggttggccagccttgccttgataaaggcaaacttgaccaatagtcttagagtatccagtgagg

SEQ ID NO: 60 - DNA, 86 bp, Artificial Sequence
  ssODN_21
tccccacactatctcaatgcaaatatctgtctgaaacggtagataaccctggctaaactccacccatgggttggccagccttgcct

SEQ ID NO: 61 - DNA, 84 bp, Artificial Sequence
  ssODN_22
tatcctcttgggggccccttccccacactatctcaatgcaacagaaacggtccctggctaaactccacccatgggttggccagc

SEQ ID NO: 62 - DNA, 83 bp, Artificial Sequence
  ssODN_23
cctatgcctaaaacacatttcacaatccctgaacttttcagataattggtacatgctttaactttaaactacaggcctcactg

SEQ ID NO: 63 - DNA, 84 bp, Artificial Sequence
  ssODN_24
cctatgcctaaaacacatttcacaatccctgaacttttcagataaattggtacatgctttaactttaaactacaggcctcactg

SEQ ID NO: 64 - DNA, 85 bp, Artificial Sequence
  ssODN_25
cctatgcctaaaacacatttcacaatccctgaacttttcagataaaattggtacatgctttaactttaaactacaggcctcactg

SEQ ID NO: 65 - DNA, 85 bp, Artificial Sequence
  ssODN_26
cctatgcctaaaacacatttcacaatccctgaacttttcagatagaattggtacatgctttaactttaaactacaggcctcactg

SEQ ID NO: 66 - RNA, 80 nt, Artificial Sequence
  synthetic
guuuuagagcuagaaauagcaaguuaaaauaaggcuaguccguuaucaacuugaaaaaguggcaccgagucggugcuuuu

SEQ ID NO: 67 - DNA, 24 bp, Artificial Sequence
  synthetic
ggactatcatatgcttaccgtaac

SEQ ID NO: 68 - DNA, 20 bp, Artificial Sequence
  synthetic
ggcgggccatttaccgtaag

SEQ ID NO: 69 - RNA, 100 nt, Artificial Sequence
  sgRNA-1
acuggauacucuaagacuauguuuuagagcuagaaauagcaaguuaaaauaaggcuaguccguuaucaacuugaaaaaguggcaccgagucggugcuuuu

SEQ ID NO: 70 - RNA, 100 nt, Artificial Sequence
  sgRNA-2
cuugucaaggcuauuggucaguuuuagagcuagaaauagcaaguuaaaauaaggcuaguccguuaucaacuugaaaaaguggcaccgagucggugcuuuu

SEQ ID NO: 71 - RNA, 100 nt, Artificial Sequence
  sgRNA-3
guuugccuugucaaggcuauguuuuagagcuagaaauagcaaguuaaaauaaggcuaguccguuaucaacuugaaaaaguggcaccgagucggugcuuuu

SEQ ID NO: 72 - RNA, 100 nt, Artificial Sequence
  sgRNA-4
uggucaaguuugccuugucaguuuuagagcuagaaauagcaaguuaaaauaaggcuaguccguuaucaacuugaaaaaguggcaccgagucggugcuuuu

SEQ ID NO: 73 - RNA, 100 nt, Artificial Sequence
  sgRNA-5
cuugaccaauagccuugacaguuuuagagcuagaaauagcaaguuaaaauaaggcuaguccguuaucaacuugaaaaaguggcaccgagucggugcuuuu

SEQ ID NO: 74 - RNA, 100 nt, Artificial Sequence
  sgRNA-6
uaucugucugaaacggucccguuuuagagcuagaaauagcaaguuaaaauaaggcuaguccguuaucaacuugaaaaaguggcaccgagucggugcuuuu

SEQ ID NO: 75 - RNA, 100 nt, Artificial Sequence
  sgRNA-7
augcaaauaucugucugaaaguuuuagagcuagaaauagcaaguuaaaauaaggcuaguccguuaucaacuugaaaaaguggcaccgagucggugcuuuu

SEQ ID NO: 76 - RNA, 100 nt, Artificial Sequence
  sgRNA-8
ucccugaacuuuucaaaaauguuuuagagcuagaaauagcaaguuaaaauaaggcuaguccguuaucaacuugaaaaaguggcaccgagucggugcuuuu

SEQ ID NO: 77 - DNA, 22 bp, Homo sapiens
tactgcgctgaaactgtggctt

SEQ ID NO: 78 - DNA, 22 bp, Homo sapiens
cttcccagggtttctcctccag

SEQ ID NO: 79 - DNA, 26 bp, Homo sapiens
tgcactgaaactgttgctttatagga

(19) United States
(12) Patent Application Publication
(10) Pub. No.: US 2023/0310506 A1
(43) Pub. Date: Oct. 5, 2023

(54) METHOD FOR ACTIVATING EXPRESSION OF GAMMA-GLOBIN GENE, AND COMPOSITION

(71) Applicant: GUANGZHOU REFORGENE MEDICINE CO., LTD., Guangzhou, Guangdong (CN)

(72) Inventors: Junbin LIANG, Guangzhou (CN); Bo GU, Guangzhou (CN); Hui XU, Guangzhou (CN)

(21) Appl. No.: 18/010,448

(22) PCT Filed: Jul. 14, 2020
(86) PCT No.: PCT/CN2020/101936

§ 371 Date: Dec. 14, 2022

(30) Foreign Application Priority Data
Jul. 1, 2020 (CN) 202010616648.7

(51) Int. Cl. A61K 35/28; A61K 48/00; A61K 38/42; C12N 15/11; C12N 9/22; C12N 15/90; C12N 15/113; C12N 15/85; C12N 5/0789

(52) U.S. Cl. CPC A61K 35/28; A61K 48/0066; A61K 38/42; C12N 15/11; C12N 9/22; C12N 15/907; C12N 15/113; C12N 15/85; C12N 5/0647; C12N 2310/315; C12N 2310/20; C12N 2800/80; C12N 2800/107; C12N 2830/001

(57) ABSTRACT

Provided is a new method for activating transcription of a gamma-globin gene. The method uses a single-stranded oligonucleotide (ssODN) containing GATA or an antisense complementary sequence TATC thereof as guidance information, and performs gene editing in a gamma-globin gene promoter region to form a GATA-containing enhancer element, which can promote the expression of the gamma-globin gene in mature red blood cells. Hematopoietic stem cells genetically edited by the method have normal functions, can significantly improve the expression of fetal hemoglobin after being differentiated into red blood cells, and therefore can be used in clinical treatment of beta-thalassemia and sickle cell anemia.

### CROSS REFERENCE TO RELATED APPLICATIONS

This application is a National Stage entry application of PCT International Application No. PCT/CN2020/101936, filed with the China National Intellectual Property Administration (CNIPA) on Jul. 14, 2020. International Application No. PCT/CN2020/101936 claims priority to and benefits of Chinese Patent Application No. 202010616648.7, filed with the CNIPA on Jul. 1, 2020. The entire contents of all of the above-identified applications are incorporated herein by reference.

### SEQUENCE LISTING

The instant application contains a Sequence Listing which is being submitted in ASCII format via EFS-WEB and is hereby incorporated by reference in its entirety. Said ASCII copy, created on Dec. 13, 2022, is named 42YV363668US_ST25.txt and is 20,487 bytes in size.

### TECHNICAL FIELD

The present disclosure relates to the technical field of gene editing technology, and relates to a method, composition and application for activating γ-globin gene expression.

### BACKGROUND

Hemoglobin (Hb for short) is a special protein that carries and transports oxygen in erythrocytes. Hemoglobin is composed of globin and heme. Hemoglobin in adults is mainly a tetramer (α2 β2) composed of 2 α-globins and 2 β-globins, which becomes an adult hemoglobin (HbA). Mutations in the β-globin gene (HBB) can cause β-hemoglobin disorders (also known as β-hemoglobinopathies), including sickle cell disease (SCD) and β-thalassemia ((β-thal). Sickle cell anemia is caused by point mutations in the structural gene of β-globin, resulting in the production of abnormal hemoglobins (HbS). β-thalassemia is caused by partial or complete defects in the β-globin gene expression, resulting in deficiency or absence of HbA. The reduction or absence of the globin chains of hemoglobins can lead to abnormal hemoglobin structures, the erythrocytes containing the same have reduced deformability, shortened lifespan, and may initiate in situ hemolysis while in the bone marrow, and may be destroyed in advance by the visceral organs such as spleen after entering the peripheral blood circulation, resulting in anemia, iron accumulation in the body and even dysplasia. Hemoglobinopathies affect millions of people around the world. Currently, approximately 330,000 children are born with hemoglobinopathies each year, which threatens human health and even life seriously. Patients with thalassemia and sickle cell disease are mainly relieved by standardized long-term blood transfusion and iron chelation therapy, which are not only incurable, but also having high safety risks. Allogeneic hematopoietic stem cell transplantation is the only treatment technology that can cure thalassemia and sickle cell disease currently. However, it is not able to be applied clinically in large scale for the limitations brought by factors such as low success rate of bone marrow matching and risk of immune rejection. Therefore, there is an urgent need to develop novel treatments which are safe and effective.

During human development, hemoglobin is not always composed of two α-globins and two β-globins. During embryonic development and shortly after birth, hemoglobin exists in the form of a tetramer (α2γ2) composed of two α-globin chains and two γ-globin chains, called fetal hemoglobin (HbF), which has a stronger oxygen affinity than HbA. With the development of fetus, the γ-globin is gradually silenced and not expressed, while the expression of β-globin gene near the same genomic site is gradually increased. About half a year after birth, the composition and proportion of hemoglobins in the blood gradually become stable, and HbF is replaced by adult hemoglobin (HbA), with only extremely low level of HbF (less than 1% of total hemoglobins) remained.

Studies have found that some populations have mutations at specific sites where the transcription of the γ-globin gene is activated, resulting in an increase in the proportion of HbF in hemoglobins, which is called hereditary persistence of fetal hemoglobin (HPFH). For example, some different types of mutations were found in the γ-globin gene promoter region, such as −114 to −102 13 bp del c., 4 bp del c.−225 to −222, c.−114C>T, c.−117G>A, c.−158C>T, c.−167C>T, c.−170>G>A, c.−175T>G, c.−175T>C, c.−195C>G, c.−196C>T, c.−198T>C, c.−201C>T, etc. These naturally occurring mutations increase the proportion of HbF in total hemoglobins to different extents. Clinical studies have found that in few patients with β-thalassemia or sickle cell anemia, the symptoms of anemia are reduced or relieved to a certain extent or the need for blood transfusions is reduced due to that the expression of HbF makes up the shortage of HbA resulting from the apropos presence of HPFH mutations in their genomes. Inspired by this finding, scientists continue to explore various ways to induce HbF expression to achieve the purpose of treating β-hemoglobinopathies. For example, hydroxyurea and other drugs are currently used clinically to induce HbF expression to treat β-hemoglobinopathies. However, the level of induced HbF in most patients is too low to completely improve the patient's condition.

Gene editing technology brings new hope and new methods for the treatments of genetic diseases such as hemoglobinopathies. In recent years, the breakthrough development of gene editing technology has made it possible and easier to artificially change the base sequence of specific sites in the genome. The gene editing technologies relatively well developed are ZFN (zinc finger nuclease), TALEN (transcription activator-like effector nuclease) and CRISPR (clustered regularly interspaced short palindromic repeats)/Cas system (CRISPR-Cas system). By designing a specific gene editing system, the target genomic DNA sites in a living cell is cleaved to form double-strand breaks (DSBs). Cells can use a non-homologous end joining (NHEJ) repair mechanism to repair DNA nicks, which can generate mutations randomly, such as sequence insertions, deletions, base substitutions, etc. If an artificially designed DNA donor template (donor template) is provided at the same time as gene editing, cells can use homology-directed repair (HDR) mechanism to complete the repair, thereby introducing the expected base mutation form at the target site.

Currently, there are several different approaches internationally for the therapeutic strategy of enhancing HbF expression in erythrocytes by gene editing. (1) Delete or destroy the erythroid enhancer element in the intron of BCL11A gene on human chromosome 2, and the BCL11A expression in erythrocytes is reduced, thereby the transcriptional inhibitory effect of BCL11A on the γ-globin gene is relieved. (2) Delete or destroy some sequences in the γ-globin gene promoter region to prevent the binding of transcriptional repressors, or introduce naturally occurring HPFH mutation types, such as 13 base pairs deletions (13 bp del c.) at site −114 to −102. (3) Generate deletion of a large fragment of DNA of 3.5 kb to 13.6 kb, and delete the sequence containing an unknown repressor between γ-globin gene and β-globin to promote the binding of γ-globin gene to the distal LCR enhancer. The approaches have shown the effect of activating HbF expression, but the long-term efficacy or/and safety of the approaches is unclear. Therefore, new approaches need to be further developed.

### SUMMARY

The present disclosure provides a method for activating γ-globin gene transcription by gene editing. The present disclosure uses a single-stranded oligonucleotide (ssODN) containing effective active GATA element or corresponding antisense complementary sequence, to generate a few base mutations (deletions, substitutions, insertions, etc.) in the proper position of the γ-globin gene promoter region by gene editing technology (such as CRISPR-Cas gene editing system). After editing, an erythroid enhancer containing the effective active GATA element is formed on the sense or antisense strand of the γ-globin gene promoter region, the effective active GATA element includes the sequence structure of NTG-N(7-8)-WGATAR or NAG-N(7-8)-WGATAR. It can promote the transcription of γ-globin gene in mature erythrocytes and increase HbF expression in erythrocytes. Herein, W is T or A; R is A or G; N is A, G, C or T, preferably C or T; N(7-8) refers to 7 to 8 random bases.

As shown in FIG. 1, the principle of the present disclosure is to conduct efficient gene editing in a stem cell or a progenitor cell (such as CD34+ hematopoietic stem cell, etc.) with erythroid differentiation ability based on gene editing technology such as CRISPR-Cas, combined with the single-stranded oligonucleotide (ssODN) designed in the present disclosure,. Mutations are made in the promoter of HBG1 and HBG2 genes (located on human chromosome 11) encoding human γ-globin, and a sequence structure of NTG-N(7-8)-WGATAR or NAG-N(7-8)-WGATAR is artificially created on the sense or antisense strand of the promoter. The sequence acts as an enhancer and can recruit activating factors such as GATA1 and TAL1 to promote γ-globin expression after the target cell differentiated into erythrocyte.

The present disclosure provides an ssODN containing NTG-N(7-8)-WGATAR sequence, NAG-N(7-8)-WGATAR sequence or reverse complementary sequence thereof, which can be used for gene editing of γ-globin gene. In the present disclosure, the ssODN structure comprises a 5′ homology arm, a substitution sequence (used to substitute a base sequence of HBG1 or HBG2 gene) and a 3′ homology arm. Herein, the GATA or TATC sequence in NTG-N(7-8)-WGATAR sequence, NAG-N(7-8)-WGATAR sequence or reverse complementary sequence thereof of ssODN structure can be located at any position in the ssODN, including the substitution sequence, the 5′ homology arm, the 3′ homology arm, the junction of the 5′ homology arm and the substitution sequence, and the junction of the 3′ homology arm and the substitution sequence. The length of a unilateral homology arm could be 20 to 300 nt. In the embodiments of the present disclosure, the length of the ssODN homology arm is about 40 nt. In some embodiments of the present disclosure, the ssODN is a sequence selected from the group consisting of SEQ ID NO: 40 to SEQ ID NO: 65. In some embodiments thereof, the 5′ and 3′ of the ssODN are phosphorothioate modified.

The present disclosure discloses a composition for use in γ-globin gene editing, containing ssODN, sgRNA and CRISPR-Cas nuclease. In some embodiments of the present disclosure, the site targeted by sgRNA of the gene editing system is located in the sequences shown in any one of SEQ ID NO: 32 to SEQ ID NO: 39. In some embodiments of the present disclosure, the sgRNA is the sequence shown in any one of SEQ ID NO: 69 to SEQ ID NO: 76. In some embodiments of the present disclosure, the Cas9 protein is a Cas 9 protein derived from Streptococcus pyogenes.

In the present disclosure, gRNA (guide RNA) is a RNA capable of binding and guiding a nuclease, such as sgRNA (single guide RNA).

The disclosure also discloses an electroporation method for use in γ-globin gene editing. The method utilizes electroporation technology to efficiently transfect a human hematopoietic stem cell with the composition formed by ssODN, sgRNA, and CRISPR-Cas nuclease to mediate efficient gene editing.

The disclosure also discloses a hematopoietic stem cell. The composition comprising ssODN, sgRNA and CRISPR-Cas nuclease is transferred into the hematopoietic stem cell by electroporation method. The γ-globin gene promoter region in the cell contains active GATA element, which could increase the γ-globin gene expression during erythroid differentiation.

Compared with the prior art, the present disclosure has the following beneficial effects:

The ssODN of the present disclosure is introduced into cells together with a gene editing system (such as CRISPR-Cas, TALEN, ZFN, etc.), which guides the gene editing of the γ-globin gene promoter region, and mutate at specific sites to form GATA element (such as NTG-N(7-8)-WGATAR sequence or NAG-N(7-8)-WGATAR sequence) with an effective activating effect. In some embodiments of the present disclosure, the ssODN and the CRISPR-Cas gene editing system are introduced into human hematopoietic stem cells by electroporation technology, and efficient gene editing is achieved in the γ-globin gene promoter region. Mutations are introduced at specific sites to form the expected erythroid enhancer element containing NTG-N(7-8)-WGATAR sequence or NAG-N(7-8)-WGATAR sequence. After the hematopoietic stem cells differentiate into erythrocytes, the γ-globin gene expression significantly increases and the proportion of HbF in hemoglobin significantly increases.

### BRIEF DESCRIPTION OF THE DRAWINGS

FIG. 1 illustrates the technical principle of the present disclosure.

FIG. 2A shows that the gene editing targeting region is the wild-type sequence between −92 and −66 of HBG1 and HBG2 genes, also shows the position of the gRNA targeting sequence used and the mutated sequence expected after gene editing.

FIG. 2B shows that the gene editing targeting region is the wild-type sequence between −129 and −98 of HBG1 and HBG2 genes, also shows the position of the gRNA targeting sequence used and the mutated sequence expected after gene editing.

FIG. 2C shows that the gene editing targeting region is the wild-type sequence between −175 and −153 of HBG1 and HBG2 genes, also shows the position of the gRNA targeting sequence used and the mutated sequence expected after gene editing.

FIG. 2D shows that the gene editing targeting region is the wild-type sequence between −192 and −160 of HBG1 and HBG2 genes, also shows the position of the gRNA targeting sequence used and the mutated sequence expected after gene editing.

FIG. 2E shows that the gene editing targeting region is the wild-type sequence between −428 and −406 of HBG1 gene and −432 and −410 of HBG2 gene, also shows the position of the gRNA targeting sequence used and the mutated sequence expected after gene editing.

FIG. 3 shows the editing efficiency data of HBG1 and HBG2 genomes by transfection of CRISPR-Cas plasmids in 293T cells. The data comes from the INDEL detection results of genomic DNA four days after the transfection of CRISPR-Cas plasmids in 293T cells using Lipofectamine 2000™ The figure shows the percentage of total indel mutations (% of total mutations).

FIG. 4 shows the editing efficiency data of HBG2 genome by simultaneous transfection of CRISPR-Cas plasmid and ssODN in 293T cells. The data comes from the INDEL detection results of HBG2 four days after the transfection of CRISPR-Cas plasmid and ssODN in 293T cells using Lipofectamine 2000™.

FIG. 5 shows the editing efficiency data of HBG2 genome by simultaneous transfection of CRISPR-Cas plasmid and ssODN in K562 cells. The data comes from the INDEL detection results of HBG2 four days after the transfection of CRISPR-Cas plasmid and ssODN in K562 cells using Lonza 4D electroporation.

FIG. 6 shows the editing efficiency data of HBG2 genome after transduction of ribonucleoprotein (RNP) and ssODN in K562 cells by electroporation. The data comes from the INDEL detection results of HBG2 four days after the transduction of ssODN and CRISPR-Cas/sgRNA RNP in K562 cells using electroporation.

FIGS. 7A-7C shows the editing efficiency data of HBG2 gene after transduction of RNP and ssODN in mobilized peripheral blood CD34+ cells (mPBSCs) by electroporation. The data comes from the INDEL detection results of genomic DNA 5 days after the induction of erythroid differentiation followed by the transduction of ssODN and CRISPR-Cas/sgRNA RNP in mPBSCs by electroporation. FIGS. 7A and 7B are Sanger sequencing maps, and FIG. 7C is the INDEL ratio result after analysis by synthego software.

FIG. 8 shows the γ-globin mRNA transcription data after gene editing of mobilized peripheral blood CD34+ cells (mPBSCs). The data comes from the qRT-PCR detection result of mRNA 18 days after the induction of erythroid differentiation followed by the transduction of ssODN and CRISPR-Cas/sgRNA RNP in mPBSCs by electroporation. The expression level of GAPDH is set as the internal reference to the γ-globin mRNA expression in the figure, and the γ-globin mRNA expression was normalized with the γ-globin mRNA expression in cells without editing as a reference, n=3.

FIG. 9 shows the HbF expression data after gene editing of mobilized peripheral blood CD34+ cells (mPBSCs). The data comes from HPLC detection result of hemoglobin after 18 days after the induction of erythroid differentiation followed by the transduction of ssODN and CRISPR-Cas/sgRNA RNP in mPBSCs by electroporation. HbF expression in the figure is the percentage of HbF in total hemoglobin.

### DETAILED DESCRIPTION OF THE EMBODIMENTS

In order to facilitate understanding of the present disclosure, the present disclosure will be described more comprehensively with reference to the related drawings as follows. Preferred embodiments of the disclosure are shown in the drawings but the present disclosure may be embodied in many different forms and is not limited thereto. On the contrary, the purpose of providing these embodiments is to understand the present disclosure thoroughly and comprehensively.

Unless otherwise defined, all technical and scientific terms used herein have the same meaning as commonly understood by the skilled in the art. The terms used herein in the description of the present disclosure are for the purpose of describing specific embodiments only and are not intended to limit the present disclosure. As used herein, the term “and/or” includes any and all combinations of one or more of the associated listed items.

The raw materials used in the following examples are all commercially available.

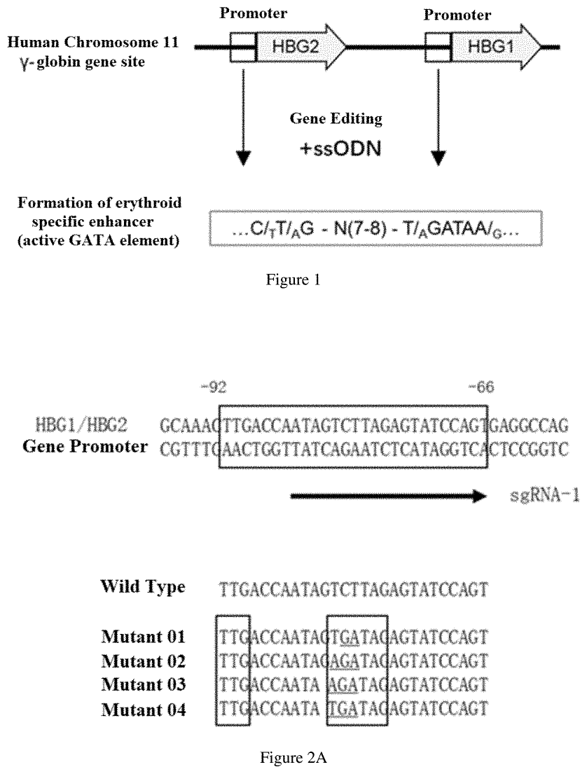

The disclosure provides by using of gene editing technology (such as CRISPR-Cas-mediated gene editing technology), combined with the single-stranded oligonucleotides (ssODN) designed by the present disclosure, performing efficient gene editing on stem cells and progenitor cells with erythroid differentiation ability such as CD34+ hematopoietic stem cells. Mutations are made in the promoter of HBG1 and HBG2 genes (located on human chromosome 11) encoding human γ-globin, and a sequence structure of NTG-N(7-8)-WGATAR or NAG-N(7-8)-WGATAR is artificially created on the sense or antisense strand of the promoter (FIG. 1). The sequence acts as an enhancer and can recruit activating factors such as GATA1 and TAL1 to promote γ-globin expression after the target cells differentiated into erythrocytes.

GATA-1 is a transcriptional regulator that is specifically and highly expressed in the erythroid system and plays an important role in the process of erythroid differentiation. Genes that are specifically highly expressed in erythrocytes, such as α- and β-globin and heme biosynthetic enzymes, are directly activated by GATA-1 transcription. As a transcription factor, GATA-1 selectively binds to WGATAR sequence element on chromatin DNA (wherein W represents T or A, R represents A or G, and can also be represented by T/A(GATA)A/G; the antisense complementary sequence is YTATCW, wherein Y represents T or C, and can also be represented by T/C(TATC)T/A). It can be seen that the “antisense complementary” used herein refers to “reverse complementary” known in the art. For example, multiple GATA sites have also been found in the promoter region of the human β-globin gene. Notably, not all genes containing GATA sequence would be activated by GATA-1. This is because GATA sequence is widely distributed in the genome and often act with a need to form a certain form of sequence combination (element combination) with upstream and downstream sequences (cis elements). For example, there are cis-regulatory elements such as CACCC or GC box near the GATA sequence, which usually recruit GATA-1 to bind together to the transcription factor EKLF. For example, there is a E-box element (CAGNTG, wherein N represents any base) or half E-box element (NTG) near GATA sequence, which recruits GATA-1 to bind together to the transcription fact TALE ChIP-seq studies on the binding sites of GATA-1 and TAL1 at the genome level showed that about three-quarters of the GATA-1/TAL1 co-binding sites have a obvious sequence regularity, that there is often a half E-box element (NTG) which is 7-8 bases upstream of the WGATAR sequence, which can be represented by NTG-N(7-8)-WGATAR. Experiments have shown that this sequence is an active GATA element that promotes some gene expression in erythrocytes. For example, the +58 enhancer in the BCL11A intron contains the CTG-N(7)-TGATAA sequence, and the disruption of the GATA motif in this sequence can lead to a significantly reduction of BCL11A expression in erythrocytes.

The sequences of 1400 bases upstream of the transcription start site of HBG1 and HBG2 genes have an extremely high similarity and are considered to be the promoter region. The inventor of the present disclosure has found from studies that the promoter region of the γ-globin itself contains multiple GATA sequence (or TATC) and a large number of TG sequence, but γ-globin is hardly expressed in mature erythrocytes. The inventor of the present disclosure has found through profound research that GATA (or TATC) and TG sequences of the γ-globin gene promoter do not form effective active GATA elements in position and direction (such as TG-N(7-8)-WGATAR), and therefore cannot recruit transcription activators such as GATA-1/TAL1 that are highly expressed in mature erythrocytes to bind (co-bind) to the promoter region of the γ-globin gene, so γ-globin is silenced and is hardly expressed in mature erythrocytes.

Through profound research, the inventor of the present disclosure found that there are some sites in the promoter regions of HBG1 and HBG2, the sequences of the sense or antisense strands of which can form the sequence structure of NTG-N(7-8)-WGATAR or NAG-N(7-8)-WGATAR by changes (such as substitutions, deletions, insertions, etc.) of a few bases. The inventor of the present disclosure consider that it is possible to edit a certain proportion of the genome to an expected sequence structure if a gene editing system is used to cleave the sites to form DSBs, by adding a donor DNA template (such as a single-stranded oligonucleotide ssODN) to guide the sites for HDR repair. Finally, the γ-globin gene promoter region, can act as an enhancer to recruit highly-expressed transcription activators in mature erythrocytes such as GATA-1/TAL1, etc. to bind (co-bind) to the promoter region of the γ-globin gene due to the formation of the sequence such as NTG-N(7-8)-WGATAR or NAG-N(7-8)-WGATAR, thereby promotes the high expression of γ-globin in mature erythrocytes.

Gene editing can be divided into two categories in terms of purposes. One is to destroy the sequence or structure of the target site, which is called knock out. The purpose of another type is to introduce specific sequence mutation at the target site, which is called knock in. To introduce specific sequence mutations, the most common method is to introduce a DNA donor template at the same time, and cells can use the mechanism of homologous recombination to replace the sequence in the template in the genome to achieve knock-in. DNA templates for HDR can be plasmid DNA, linearized double-stranded DNA, AAV-delivered DNA, or single-stranded oligonucleotides (ssODN). ssODN is used more and more in gene editing, as it has high efficiency and is easy to synthesis, which is particularly suitable for gene editing in cultured cells in vitro.

The ssODN structure contains a 5′ homology arm, a substitution sequence, and a 3′ homology arm. Homology arms are sequences that are homologous to the DNA regions flanking the target site intended to be edited, and are used to locate the target site on the chromosome. The corresponding sequences of the 5′ homology arm and the 3′ homology arm on the chromosome are usually discontinuous in position, with a certain space among them, such as an spacer of 1-20 nucleotides, and these spacer sequences are the targets (intended-editing sequence) of gene editing, i.e., the sequence that is desired to be substituted by gene editing. The “substitution sequence” between the 3′ terminus of the 5′ homology arm and the 5′ terminus of the 3′ homology arm on the ssODN is the desired result of gene editing. It is able to perform homologous recombination repair while inducing gene editing by adding ssODN while performing gene editing. As a result of the repair, the intended-editing sequence in the cell genome is repaired into a substitution sequence. The length of the substitution sequence in the ssODN can be shorter than that of the intended-editing sequence, in which case the result of gene editing is the deletion or/and substitution of the intended-editing sequence in the original genome. The length of the substitution sequence in the ssODN can be 0 base, in which case the intended-editing sequence on the genome is deleted. The length of the substitution sequence in the ssODN may be longer than that of the intended-editing sequence, in which case the result of gene editing is that the intended-editing sequence is replaced or/and sequence insertion.

The present disclosure is suitable for gene editing systems mediated by site-specific nucleases such as “CRISPR-Cas”, TALEN and ZFN. “CRISPR-Cas” is a gene editing technology, including but not limited to various natural or artificially designed CRISPR-Cas systems, such as the CRISPR-Cas9 system, CRISPR-Cas12 system, etc. The working principle of CRISPR-Cas9 is that crRNA (CRISPR-derived RNA) combines with tracrRNA (trans-activating RNA) through base pairing to form a tracrRNA/crRNA complex, which guides the nuclease Cas9 protein to cleave double-stranded DNA at the sequence target sites paired with crRNA. The role of tracrRNA and crRNA can also be substituted by an artificially synthesized sgRNA with a guiding role. When using other CRISPR-Cas systems, it is necessary to design the corresponding sgRNA or crRNA. When using systems such as TALEN or ZFN, the corresponding TALEN or ZFN nuclease needs to be designed according to the editing site disclosed in the present disclosure.

### Example 1: Screening of ssODN and CRISPR-Cas Systems for the Introduction of an Active GATA Element into the γ-Globin Gene Promoter

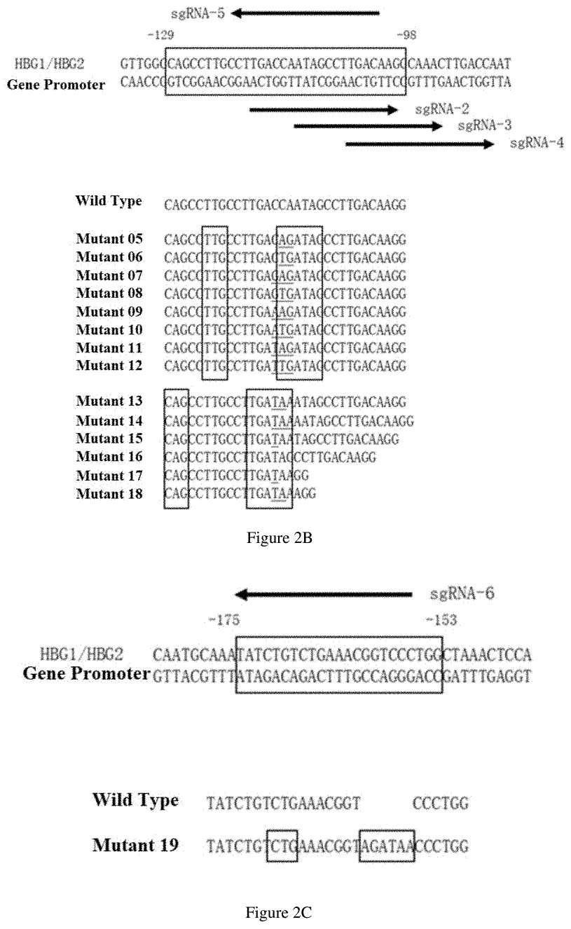

By analyzing the sequences (SEQ ID NO: 1 and SEQ ID NO: 2) within about 1.4 kb upstream of the transcription start sites of HBG1 and HBG2, the inventor found that the sequences of many segments satisfy that forming the sequence structure of NTG-N(7-8)-WGATAR or NAG-N(7-8)-WGATAR by changes of a few bases such as substitution, deletion, insertion, etc.. By analyzing whether there is an NGG (PAM recognized by spCas9) sequence near the target mutant base in these segments and whether the cleavage site is on target of the target sequence to be edited, five candidate targeting regions most likely to achieve efficient gene editing were finally obtained (FIGS. 2A to 2E). According to the distance from the transcription start site, they are: Region 1, between −92 to −66 of HBG1 and HBG2 promoters (FIG. 2A); Region 2, between −129 to −98 of HBG1 and HBG2 promoters (FIG. 2B); Region 3, between −175 and −153 of HBG1 and HBG2 promoters (FIG. 2C); Region 4, between −192 and −160 of HBG1 and HBG2 genes (FIG. 2D); Region 5, between −428 to −406 of HBG1 gene and −432 to −410 of HBG2 gene (FIG. 2E).

According to the sequence features of the five candidate regions and the predicted cleavage site position of the CRISPR-Cas9 gene editing system, the suitable mutation target types in each region is obtained by analyzing, as is shown in Table 1 (SEQ ID NO: 3 to SEQ ID NO: 31). The underline herein indicates the base that needs to be substituted, which is the desired result after gene editing. In the present disclosure, the length of the substitution base is 0-6 bases, and the effect achieved is deletion, substitution, insertion, or a combination of deletion, substitution, and insertion.

TABLE 1
Wild-type sequences of HBG1 and HBG2 promoters
and sequences obtained after editing

| | | Target site and edited |
|---|---|---|
| SEQ ID | Number | sequence (5′-3′) |
| SEQ ID NO: 3 | Region 1, | TTGACCAATAGTCTTAGAGTATCCAGT |
| | wild-type | |
| SEQ ID NO: 4 | Mutant_01 | TTGACCAATAGTGATAGAGTATCCAGT |
| SEQ ID NO: 5 | Mutant_02 | TTGACCAATAGAGATAGAGTATCCAGT |
| SEQ ID NO: 6 | Mutant_03 | TTGACCAATAAGATAGAGTATCCAGT |
| SEQ ID NO: 7 | Mutant_04 | TTGACCAATATGATAGAGTATCCAGT |
| SEQ ID NO: 8 | Region 2, | CAGCCTTGCCTTGACCAATAGCCTTGACAAGG |
| | wild-type | |
| SEQ ID NO: 9 | Mutant_05 | CAGCCTTGCCTTGACAGATAGCCTTGACAAGG |
| SEQ ID NO: 10 | Mutant_06 | CAGCCTTGCCTTGACTGATAGCCTTGACAAGG |
| SEQ ID NO: 11 | Mutant_07 | CAGCCTTGCCTTGAGAGATAGCCTTGACAAGG |
| SEQ ID NO: 12 | Mutant_08 | CAGCCTTGCCTTGAGTGATAGCCTTGACAAGG |
| SEQ ID NO: 13 | Mutant_09 | CAGCCTTGCCTTGAAAGATAGCCTTGACAAGG |
| SEQ ID NO: 14 | Mutant_10 | CAGCCTTGCCTTGAATGATAGCCTTGACAAGG |
| SEQ ID NO: 15 | Mutant_11 | CAGCCTTGCCTTGATAGATAGCCTTGACAAGG |
| SEQ ID NO: 16 | Mutant_12 | CAGCCTTGCCTTGATTGATAGCCTTGACAAGG |
| SEQ ID NO: 17 | Mutant_13 | CAGCCTTGCCTTGATAAATAGCCTTGACAAGG |
| SEQ ID NO: 18 | Mutant_14 | CAGCCTTGCCTTGATAAAATAGCCTTGACAAGG |
| SEQ ID NO: 19 | Mutant_15 | CAGCCTTGCCTTGATAATAGCCTTGACAAGG |
| SEQ ID NO: 20 | Mutant_16 | CAGCCTTGCCTTGATAGCCTTGACAAGG |
| SEQ ID NO: 21 | Mutant_17 | CAGCCTTGCCTTGATAAGG |
| SEQ ID NO: 22 | Mutant_18 | CAGCCTTGCCTTGATAAAGG |
| SEQ ID NO: 23 | Region 3, | TATCTGTCTGAAACGGTCCCTGG |
| | wild-type | |
| SEQ ID NO: 24 | Mutant_19 | TATCTGTCTGAAACGGTAGATAACCCTGG |
| SEQ ID NO: 25 | Region 4, | CACTATCTCAATGCAAATATCTGTCTG |
| | wild-type | AAACGG |
| SEQ ID NO: 26 | Mutant_20 | CACTATCTCAATGCAACAGAAACGG |
| SEQ ID NO: 27 | Region 5, | TCCCTGAACTTTTCAAAAATTGG |
| | wild-type | |
| SEQ ID NO: 28 | Mutant_21 | TCCCTGAACTTTTCAGATAATTGG |
| SEQ ID NO: 29 | Mutant_22 | TCCCTGAACTTTTCAGATAAATTGG |
| SEQ ID NO: 30 | Mutant_23 | TCCCTGAACTTTTCAGATAAAATTGG |
| SEQ ID NO: 31 | Mutant_24 | TCCCTGAACTTTTCAGATAGAATTGG |

In order to cleave the DNA double-strand near the editing site of regions shown in Table 1 to form DSB, the spCas9 CRISPR-Cas system with higher cleavage efficiency (derived from Streptococcus pyogenes) is selected in the present disclosure to analyze the target sites with higher cleavage efficiency. Site_1 to site_8 were selected as candidate target sites. The identified target site of DNA sequences and PAM sequences (NGG) are shown in Table 2 (SEQ ID NO: 32 to SEQ ID NO: 39).

TABLE 2
Target sites recognized by sgRNAs on
HBG1 and HBG2 promoters

| | | sgRNA recognition | |
|---|---|---|---|
| SEQ ID | Site | site and PAM (5′-3′) | DNA strand |
| SEQ ID NO: 32 | site_1 | ACTGGATACTCTAAGACTAT TGG | antisense strand |
| SEQ ID NO: 33 | site_2 | CTTGTCAAGGCTATTGGTCA AGG | antisense strand |
| SEQ ID NO: 34 | site_3 | GTTTGCCTTGTCAAGGCTAT TGG | antisense strand |
| SEQ ID NO: 35 | site_4 | TGGTCAAGTTTGCCTTGTCA AGG | antisense strand |
| SEQ ID NO: 36 | site_5 | CTTGACCAATAGCCTTGACA AGG | sense strand |
| SEQ ID NO: 37 | site_6 | TATCTGTCTGAAACGGTCCC TGG | sense strand |
| SEQ ID NO: 38 | site_7 | ATGCAAATATCTGTCTGAAA CGG | sense strand |
| SEQ ID NO: 39 | site_8 | TCCCTGAACTTTTCAAAAAT TGG | sense strand |

According to the expected mutation type shown in Table 1, and the DNA strand (sense strand or antisense strand) recognized by sgRNA in Table 2, the corresponding ssODN is designed to guide gene editing and repair (Table 3, SEQ ID NO: 40 to SEQ ID NO: 65). In the present disclosure, the ssODN structure comprises a 5′ homology arm, a substitution sequence and a 3′ homology arm. The GATA or TATC sequence in NTG-N(7-8)-WGATAR sequence, NAG-N(7-8)-WGATAR sequence, or corresponding reverse complementary sequence thereof of ssODN structure can be located at any position on the ssODN, including the substitution sequence, the 5′ homology arm, the 3′ homology arm, the junction of the 5′ homology arm and the substitution sequence, and the junction of the 3′ homology arm and the substitution sequence. The homology arms on both sides of the ssODN can be symmetric or asymmetric (the lengths on both sides are different). The ssODN with symmetric homology arms is uniformly used for experiments in the present disclosure for the convenience of system comparison. The length of the homology arms at both sides of ssODN is 20 to 300 nt. The ssODN with a homology arm of approximately 40 nt on both sides is used to illustrate in the examples of the present disclosure for the convenience of system comparison. The ssODN can be the sense strand or the antisense strand of the edited region DNA (the homology arm sequence is the same with the corresponding sense strand or antisense strand). In the present disclosure, the DNA strand with the same sgRNA recognition site is uniformly selected by ssODN as the ssODN template sequence. Except for the sequences of homology arms on both sides, the substitution base of the sequence of ssODN can be 0, 1, 2, 3, 4, 5 or 6 bases, and the effect achieved is deletion, substitution, insertion, or a combination of deletion, substitution, and insertion. The effect achieved can be to change more than 1 base on the genome, such as deletion or substitution of 1-20 bases. In the sequences as shown in Table 3 (SEQ ID NO: 40 to SEQ ID NO: 65), the underlined bases are homology arms on both sides with a length of about 40 nt, the non-underlined bases between the homology arms are substitution bases, wherein ssODN_16 has 0 substitution base between the homology arms on both sides. The first three nucleotides at both terminuses of ssODN were modified with phosphorothioate to enhance the stability of ssODN and improve its activity in gene editing.

TABLE 3
ssODN sequence and the matched gRNA number

| SEQ ID | ssODN | ssODN sequence (5′-3′) | Applicable area |
|---|---|---|---|
| SEQ ID | ssODN_1 | CCTAGCCAGCCGCCGGCCCCTGGCCTCACTGGATACT | Region 1 |
| NO: 40 | | CTATCACTATTGGTCAAGTTTGCCTTGTCAAGGCTAT | |
| | | TGGTCAAGGC | |
| SEQ ID | ssODN_2 | CCTAGCCAGCCGCCGGCCCCTGGCCTCACTGGATACT | Region 1 |
| NO: 41 | | CTATCTCTATTGGTCAAGTTTGCCTTGTCAAGGCTATT | |
| | | GGTCAAGGC | |
| SEQ ID | ssODN_3 | CCTAGCCAGCCGCCGGCCCCTGGCCTCACTGGATACT | Region 1 |
| NO: 42 | | CTATCTTATTGGTCAAGTTTGCCTTGTCAAGGCTATTG | |
| | | GTCAAGGC | |
| SEQ ID | ssODN_4 | CCTAGCCAGCCGCCGGCCCCTGGCCTCACTGGATACT | Region 1 |
| NO: 43 | | CTATCATATTGGTCAAGTTTGCCTTGTCAAGGCTATT | |
| | | GGTCAAGGC | |
| SEQ ID | ssODN_5 | CTCTAAGACTATTGGTCAAGTTTGCCTTGTCAAGGCT | Region 2 |
| NO: 44 | | ATCTGTCAAGGCAAGGCTGGCCAACCCATGGGTGGA | |
| | | GTTTAGCCA | |
| SEQ ID | ssODN_6 | CTCTAAGACTATTGGTCAAGTTTGCCTTGTCAAGGCT | Region 2 |
| NO: 45 | | ATCAGTCAAGGCAAGGCTGGCCAACCCATGGGTGGA | |
| | | GTTTAGCCA | |
| SEQ ID | ssODN_7 | CTCTAAGACTATTGGTCAAGTTTGCCTTGTCAAGGCT | Region 2 |
| NO: 46 | | ATCTCTCAAGGCAAGGCTGGCCAACCCATGGGTGGA | |
| | | GTTTAGCCA | |
| SEQ ID | ssODN_8 | CTCTAAGACTATTGGTCAAGTTTGCCTTGTCAAGGCT | Region 2 |
| NO: 47 | | ATCACTCAAGGCAAGGCTGGCCAACCCATGGGTGGA | |
| | | GTTTAGCCA | |
| SEQ ID | ssODN_9 | CTCTAAGACTATTGGTCAAGTTTGCCTTGTCAAGGCT | Region 2 |
| NO: 48 | | ATCTTTCAAGGCAAGGCTGGCCAACCCATGGGTGGA | |
| | | GTTTAGCCA | |
| SEQ ID | ssODN_10 | CTCTAAGACTATTGGTCAAGTTTGCCTTGTCAAGGCT | Region 2 |
| NO: 49 | | ATCATTCAAGGCAAGGCTGGCCAACCCATGGGTGGA | |
| | | GTTTAGCCA | |
| SEQ ID | ssODN_11 | CTCTAAGACTATTGGTCAAGTTTGCCTTGTCAAGGCT | Region 2 |
| NO: 50 | | ATCTATCAAGGCAAGGCTGGCCAACCCATGGGTGGA | |
| | | GTTTAGCCA | |
| SEQ ID | ssODN_12 | CTCTAAGACTATTGGTCAAGTTTGCCTTGTCAAGGCT | Region 2 |
| NO: 51 | | ATCAATCAAGGCAAGGCTGGCCAACCCATGGGTGGA | |
| | | GTTTAGCCA | |
| SEQ ID | ssODN_13 | CTCTAAGACTATTGGTCAAGTTTGCCTTGTCAAGGCT | Region 2 |
| NO: 52 | | ATTTATCAAGGCAAGGCTGGCCAACCCATGGGTGGA | |
| | | GTTTAGCCA | |
| SEQ ID | ssODN_14 | CTCTAAGACTATTGGTCAAGTTTGCCTTGTCAAGGCT | Region 2 |
| NO: 53 | | ATTTTATCAAGGCAAGGCTGGCCAACCCATGGGTGG | |
| | | AGTTTAGCCA | |
| SEQ ID | ssODN_15 | CTCTAAGACTATTGGTCAAGTTTGCCTTGTCAAGGCT | Region 2 |
| NO: 54 | | ATTATCAAGGCAAGGCTGGCCAACCCATGGGTGGAG | |
| | | TTTAGCCA | |
| SEQ ID | ssODN_16 | CTCTAAGACTATTGGTCAAGTTTGCCTTGTCAAGGCT | Region 2 |
| NO: 55 | | A | |
| | | TCAAGGCAAGGCTGGCCAACCCATGGGTGGAGTTTA | |
| | | GCCA | |
| SEQ ID | ssODN_17 | CCTCACTGGATACTCTAAGACTATTGGTCAAGTTTGC | Region 2 |
| NO: 56 | | CTTATCAAGGCAAGGCTGGCCAACCCATGGGTGGAG | |
| | | TTTAGCCA | |
| SEQ ID | ssODN_18 | CCTCACTGGATACTCTAAGACTATTGGTCAAGTTTGC | Region 2 |
| NO: 57 | | CTTTATCAAGGCAAGGCTGGCCAACCCATGGGTGGA | |
| | | GTTTAGCCA | |
| SEQ ID | ssODN_19 | TGGCTAAACTCCACCCATGGGTTGGCCAGCCTTGCCT | Region 2 |
| NO: 58 | | TGATAAGGCAAACTTGACCAATAGTCTTAGAGTATCC | |
| | | AGTGAGG | |
| SEQ ID | ssODN_20 | TGGCTAAACTCCACCCATGGGTTGGCCAGCCTTGCCT | Region 2 |
| NO: 59 | | TGATAAAGGCAAACTTGACCAATAGTCTTAGAGTATC | |
| | | CAGTGAGG | |
| SEQ ID | ssODN_21 | TCCCCACACTATCTCAATGCAAATATCTGTCTGAAAC | Region 3 |
| NO: 60 | | GGTAGATAACCCTGGCTAAACTCCACCCATGGGTTGG | |
| | | CCAGCCTTGCCT | |
| SEQ ID | ssODN_22 | TATCCTCTTGGGGGCCCCTTCCCCACACTATCTCAAT | Region 4 |
| NO: 61 | | GCAACAGAAACGGTCCCTGGCTAAACTCCACCCATG | |
| | | GGTTGGCCAGC | |
| SEQ ID | ssODN_23 | CCTATGCCTAAAACACATTTCACAATCCCTGAACTTT | Region 5 |
| NO: 62 | | TCAGATAATTGGTACATGCTTTAACTTTAAACTACAG | |
| | | GCCTCACTG | |
| SEQ ID | ssODN_24 | CCTATGCCTAAAACACATTTCACAATCCCTGAACTTT | Region 5 |
| NO: 63 | | TCAGATAAATTGGTACATGCTTTAACTTTAAACTACA | |
| | | GGCCTCACTG | |
| SEQ ID | ssODN_25 | CCTATGCCTAAAACACATTTCACAATCCCTGAACTTT | Region 5 |
| NO: 64 | | TCAGATAAAATTGGTACATGCTTTAACTTTAAACTAC | |
| | | AGGCCTCACTG | |
| SEQ ID | ssODN_26 | CCTATGCCTAAAACACATTTCACAATCCCTGAACTTT | Region 5 |
| NO: 65 | | TCAGATAGAATTGGTACATGCTTTAACTTTAAACTAC | |
| | | AGGCCTCACTG | |

The sgRNA-spCas9 vector (PX459, pSpCas9(BB)-2A-Puro) is used to express the sgRNA corresponding to the target site in Table 2. The vector expresses the spCas9 protein and can express sgRNA with a length of 100 nt. The 5′ terminus of the sgRNA is a guide sequence of 20 nt, and the latter is a general sgRNA backbone sequence of 80 nt (SEQ ID NO: 66, GUUUUAGAGCUAGAAAUAGCAAGUUAAAAUAAGGCUAGUCCGUUAUCAACUUGA AAAA GUGGCACCGAGUCGGUGCUUUU). The method of cloning sgRNA is: synthesize the corresponding guide strand sequence oligo (same as the 20nt target site sequence in Table 2) of the sgRNA and complementary strand oligo (cacc is added to the 5′-terminus of the guide strand sequence, if the first nucleotide at the 5′-terminus of the guide strand is not guanine G, then caccG is added to the 5′-terminus of the guide strand; cacc is added to the 5′-terminus of the complementary strand; if a G is added to the 5′-terminus of the guide strand, then a C is added to the 3′-terminus of the complementary strand for complementary pairing). The two oligos are mixed in equal amounts, subjected to heat deformation and annealed at 95° C., and then ligated into the PX459 vector digested with the restriction enzyme Bbs I. The success of cloning was identified by colony PCR and Sanger sequencing. The PCR was performed using SEQ ID NO: 67 (5′-3′: GGACTATCATATGCTTACCGTAAC) and SEQ ID NO: 68 (5′-3′: GGCGGGCCATTTACCGTAAG) as primers. After successfully cloning, 8 plasmids were obtained, expressing sgRNAs from SEQ ID NO: 69 to SEQ ID NO: 76, respectively (Table 4).

TABLE 4
sgRNAs expressed by PX459 vector

| SEQ ID | sgRNA | sgRNA sequence (5′-3′) |
|---|---|---|
| SEQ ID | sgRNA-1 | ACUGGAUACUCUAAGACUAU |
| NO: 69 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-2 | CUUGUCAAGGCUAUUGGUCA |
| NO: 70 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-3 | GUUUGCCUUGUCAAGGCUAU |
| NO: 71 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-4 | UGGUCAAGUUUGCCUUGUCA |
| NO: 72 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-5 | CUUGACCAAUAGCCUUGACA |
| NO: 73 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-6 | UAUCUGUCUGAAACGGUCCC |
| NO: 74 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-7 | AUGCAAAUAUCUGUCUGAAA |
| NO: 75 | | GUUUUAGAGCUAGAAAUAGCAAGUUAAAAUAAGGCUAGUCCGUUAU |
| | | CAACUUGAAAAAGUGGCACCGAGUCGGUGCUUUU |
| SEQ ID | sgRNA-8 | UCCCUGAACUUUUCAAAAAU |
| NO: 76 | | GUUUUAGAGCUAGAAAUAGCAAGUUAAAAUAAGGCUAGUCCGUUAU |
| | | CAACUUGAAAAAGUGGCACCGAGUCGGUGCUUUU |

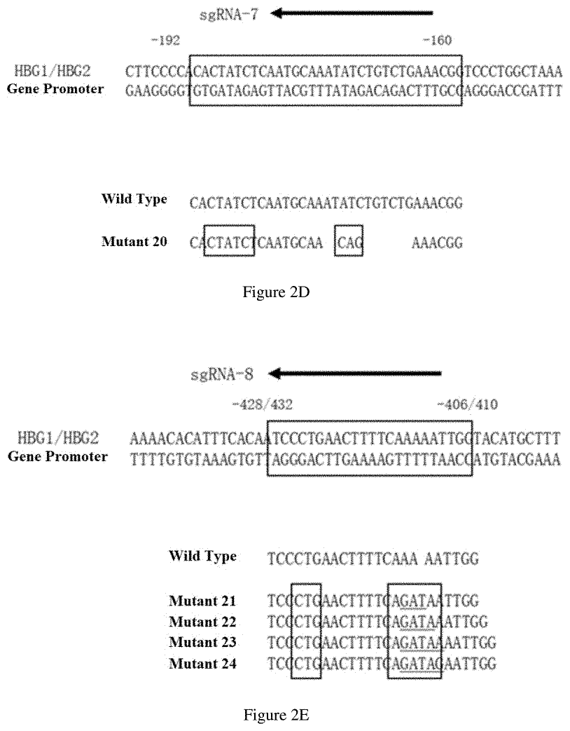

The 8 plasmids expressed were transfected into 293T cells, and the method was referred to the method of Lipofectamine 2000™ kit. The transfected cells were cultured for 4 days, and the cells were collected to extract genomic DNA. SEQ ID NO: 77 and SEQ ID NO: 78, and SEQ ID NO: 79 and SEQ ID NO: 78 were used respectively, to amplify the fragments of HBG1 and HB G2 promoter gene by KOD Plus high-fidelity enzyme PCR method. The resulting PCR products were purified and sequenced using SEQ ID NO: 77 and SEQ ID NO: 79, respectively. The files obtained from the sequencing results were analyzed by synthego software for gene editing efficiency. As shown in FIG. 3, except for sgRNA-8 with a relatively low editing efficiency (about 20%), the overall mutation (indel) efficiency of editing in other groups is about 40-50%.

TABLE 5
PCR and Sanger sequencing primers

| | | sgRNA recognition site |
|---|---|---|
| SEQ ID | site | and PAM (5′-3′) |
| SEQ ID NO: 77 | HBG1_F | TACTGCGCTGAAACTGTGGCTT |
| SEQ ID NO: 78 | HBG1/2_R | CTTCCCAGGGTTTCTCCTCCAG |
| SEQ ID NO: 79 | HBG2_F | TGCACTGAAACTGTTGCTTTAT |
| | | AGGA |

### Example 2: Co-Transfection of ssODN and sgRNA-spCas9 Vector into 293T Cells for Gene Editing

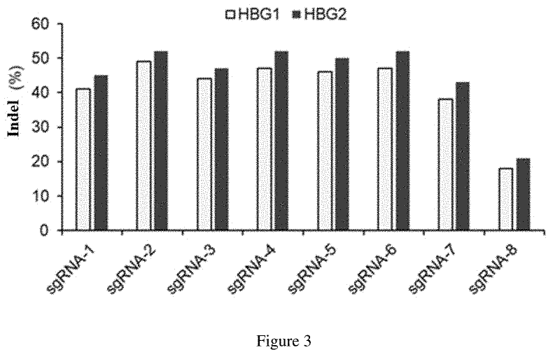

The eight sgRNA-spCas9 vectors were co-transfected with appropriate ssODN into 293T cells to test whether the addition of ssODN could induce gene editing to form the expected mutation type (occurrence of HDR repair). According to the position of the sgRNA cleavage point, the 8 sgRNA-spCas9 vectors were combined according to the sgRNA+ssODN combination method shown in Table 6 to form 30 combinations, respectively. Using Lipofectamine 2000™ reagent, ssODN was added while transfecting the plasmid, and the ssODN and the plasmid were delivered to the cells together. The transfected cells were cultured for 4 days, and the cells were collected to extract genomic DNA. Since the gene editing efficiency of HBG1 and HBG2 is almost positive correlated, and the gene editing efficiency of HBG2 is always slightly higher than that of HBG1 (FIG. 3), only the gene editing efficiency of HBG2 genome was selected for comparison in subsequent experiments. The fragment of HBG2 promoter gene was amplified by PCR, purified and sequencing according to the methods described in Example 1. The sequencing results analyzed by synthego software for gene editing efficiency, show that the addition of ssODN and the co-transfection with the sgRNA-spCas9 vector could improve the editing efficiency, and most groups induced about 10-20% of the gene editing to form the expected mutation type at the same time (FIG. 4).

TABLE 6
Co-transfection combinations of
ssODN with sgRNA-spCas9 vector

| Combination | Transfection complex |
|---|---|
| Combination 1 | sgRNA-1 + ssODN_1 |
| Combination 2 | sgRNA-1 + ssODN_2 |
| Combination 3 | sgRNA-1 + ssODN_3 |
| Combination 4 | sgRNA-1 + ssODN_4 |
| Combination 5 | sgRNA-2 + ssODN_5 |
| Combination 6 | sgRNA-2 + ssODN_6 |
| Combination 7 | sgRNA-2 + ssODN_7 |
| Combination 8 | sgRNA-2 + ssODN_8 |
| Combination 9 | sgRNA-2 + ssODN_9 |
| Combination 10 | sgRNA-2 + ssODN_10 |
| Combination 11 | sgRNA-2 + ssODN_11 |
| Combination 12 | sgRNA-2 + ssODN_12 |
| Combination 13 | sgRNA-2 + ssODN_13 |
| Combination 14 | sgRNA-2 + ssODN_14 |
| Combination 15 | sgRNA-2 + ssODN_15 |
| Combination 16 | sgRNA-2 + ssODN_16 |
| Combination 17 | sgRNA-2 + ssODN_17 |
| Combination 18 | sgRNA-2 + ssODN_18 |
| Combination 19 | sgRNA-3 + ssODN_17 |
| Combination 20 | sgRNA-3 + ssODN_18 |
| Combination 21 | sgRNA-4 + ssODN_17 |
| Combination 22 | sgRNA-4 + ssODN_18 |
| Combination 23 | sgRNA-5 + ssODN_19 |
| Combination 24 | sgRNA-5 + ssODN_20 |
| Combination 25 | sgRNA-6 + ssODN_21 |
| Combination 26 | sgRNA-7 + ssODN_22 |
| Combination 27 | sgRNA-8 + ssODN_23 |
| Combination 28 | sgRNA-8 + ssODN_24 |
| Combination 29 | sgRNA-8 + ssODN_25 |
| Combination 30 | sgRNA-8 + ssODN_26 |

### Example 3: Co-Transfection of ssODN and sgRNA-spCas9 Vector into K562 Cells by Electroporation for Gene Editing

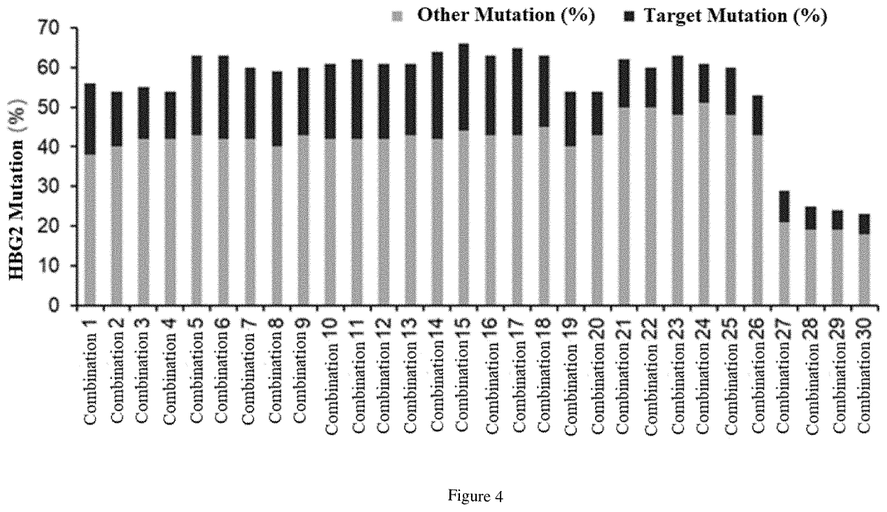

Transfection of plasmids into suspension cells using liposomes (e.g. Lipofectamine 2000™ reagent) is relatively inefficient, and transfecting suspension cells are generally introduced by electroporation. Some combinations of sgRNA and ssODN in Example 2 were taken to be introduced into K562 cells by lonza-4D electroporator, with the K562 cell transfection procedure which is built-in. The transfected cells were cultured for 4 days, and the cells were collected to extract genomic DNA. The fragment of HBG2 promoter gene was amplified by PCR to sequencing. The sequencing results analyzed by synthego software for gene editing efficiency, show that the electroporation of combinations sgRNA-1, sgRNA-2, sgRNA-5 and ssODN in K562 can achieve higher editing efficiency (FIG. 5), which is slightly higher than that in 293T cells. And the expected mutation type induced by electroporation of ssODN and sgRNA-spCas9 vector was also slightly higher than that in 293T cells.

### Example 4: Co-Transfection of ssODN and spCas9 Protein/sgRNA RNP into K562 Cells by Electroporation for Efficient Gene Editing

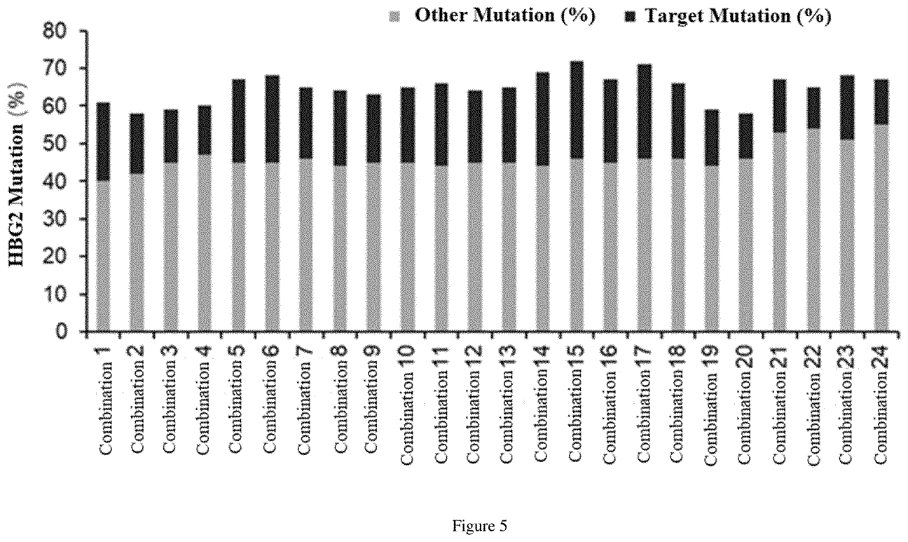

Compared with the plasmid-expressed CRISPR-Cas system, the delivery of the CRISPR-Cas system in the form of ribonucleoprotein (RNP) has been proved to have a high efficiency in gene editing and it is not easy to induce apoptosis. Commercial spCas9 protein and chemically synthesized sgRNA-1, sgRNA-2, and sgRNA-5 were used (sequences are the same as the corresponding sgRNAs in Table 4, with the terminus chemically modified) to incubate and package RNP-1, RNP-2 and RNP-5 in vitro, respectively. RNPs were delivered in combination with some ssODN into K562 cells by electroporation. The transfected cells were cultured for 4 days, and the cells were collected to extract genomic DNA. The HBG2 promoter gene fragment was amplified by PCR to conduct sequencing. The sequencing results analyzed by synthego software for gene editing efficiency, show that (FIG. 6) compared with the combination without the addition of ssODN, the addition of ssODN when introducing RNP in K562 with electroporation significantly improved the overall gene editing efficiency by about two times (FIG. 6). Meanwhile, transduction of ssODN and RNP by electroporation could induce a higher proportion of gene editing to form the expected mutation type (FIG. 6).

### Example 5: Introduction of ssODN and spCas9 Protein/sgRNA RNP into Human Hematopoietic Stem/Progenitor Cells by Electroporation for Efficient Gene Editing and Significantly Increase HBG Expression

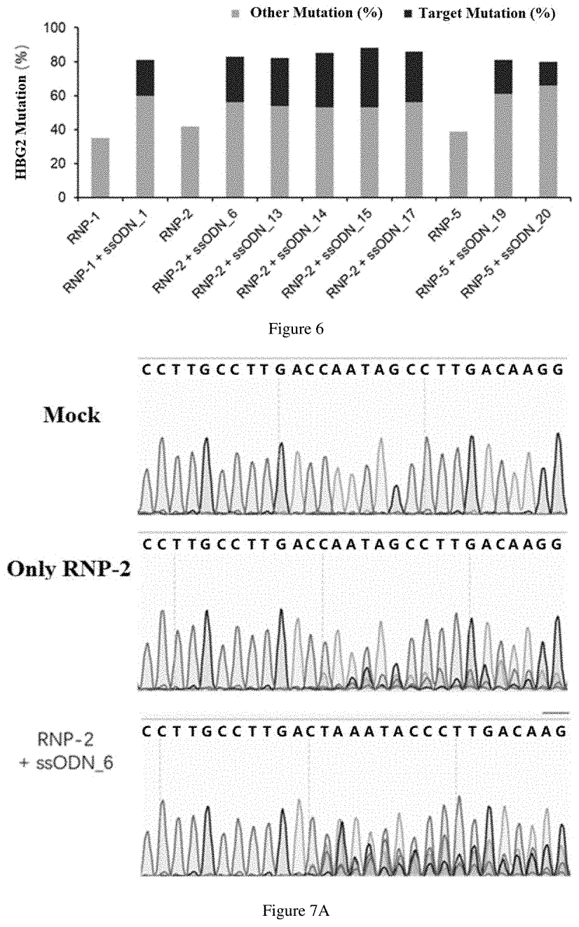

After resuscitation, mobilized peripheral blood-derived human CD34-positive cells (mPBSCs) were cultured for two days in StemSpan serum-free expansion medium (SFEM) enriched with human cytokines (SCF, TPO, Flt3L, 100 ng/ml each). ssODN and spCas9/sgRNA RNPs were delivered into mPBSCs by the EO-100 program built in the lonza-4D electroporator. After electroporation, the cells were cultured in SFEM enriched with human cytokines (SCF/TPO/F1t3L, 100 ng/ml each) for one day, and induced to differentiate erythrocytes in three stages. The first stage: culture in IMDM medium containing additives such as EPO, SCF, human AB serum, insulin, transferrin, heparin, IL-3, hydrocortisone, etc. for 7 days. On the 5th day of induced differentiation, 2×10e5 cells were taken out to extract genomic DNA, and then the HBG2 promoter fragment was amplified by PCR and sent to Sanger sequencing. Similar to the results in K562 cells, transduction of ssODN and spCas9/sgRNA RNPs by electroporation in mPBSCs resulted in efficient gene editing, with partial genome editing resulting in 30% of the expected mutation types (FIGS. 7A-7C).

Electroporated mPBSC cells and untreated mPBSC cells (control, NC) were induced for 7 days of the first stage of differentiation, and then were cultured in IMDM medium containing additives such as EPO, SCF, human AB serum, insulin, transferrin, and heparin, etc. for 4 days, and were finally cultured in IMDM medium containing additives such as EPO, human AB serum, insulin, transferrin, and heparin, etc. for 7 days. After 18 days of differentiation induction, both the electroporated mPBSCs and the untreated mPBSCs were observed dark red after centrifugation, which contained a large number of erythrocytes, indicating that the transfected mPBSCs with electroporation could differentiate into erythrocytes normally.

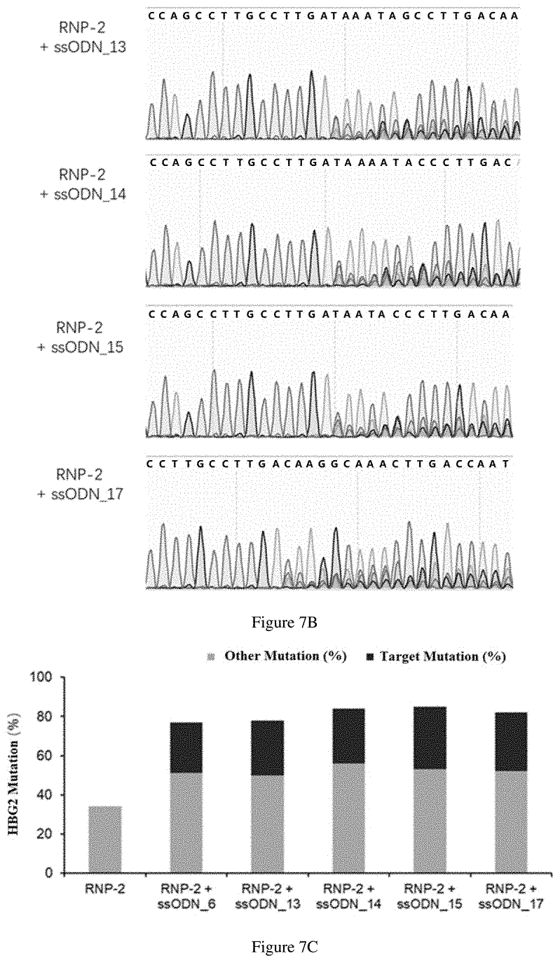

Near the end of differentiation induction, some cells were taken to extract mRNA, and the expression level of HBG mRNA was detected by reverse transcription-real-time quantitative PCR (RT-qPCR). Compared with non-transfected cells (NC), co-transfection of RNP-2 with ssODN_6, ssODN_13, ssODN_14, ssODN_15 and ssODN_17 all increased the expression level of HBG mRNA (FIG. 8), and the increase of HBG mRNA could be more than 3-fold.

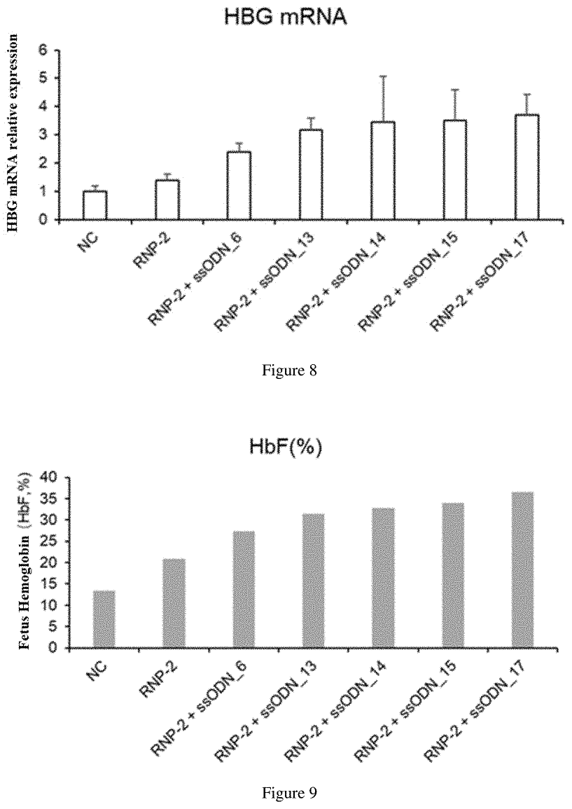

The glycosylated hemoglobin Alc detection system (VARIANT II TURBO HbA1c kit-2.0) was used to detect the hemoglobin of the erythrocytes differentiated from hematopoietic stem cells with electroporation transduction in the RNP-2 group by HPLC. The results showed that, compared with cells without gene editing, cells added with ssODN for gene editing has a significant increase in the content of fetal hemoglobin (HbF), ranging from 27% to 36%, which has more than 2-fold upregulation compared with the control group of 13.3% (FIG. 9).

1. A method for activating γ-globin gene expression, comprising the following operations: a sequence of NTG-N(7-8)-WGATAR or NAG-N(7-8)-WGATAR is artificially produced in the sense strand or antisense strand of the promoter region of γ-globin gene by gene editing technology, forming an enhancer element comprising the NTG-N(7-8)-WGATAR sequence, NAG-N(7-8)-WGATAR sequence or reverse complementary sequence thereof in the promoter region of γ-globin gene;
wherein the reverse complementary sequence of WGATAR is YTATCW, wherein W is T or A, R is A or G, Y is T or C, and N is A, G, C or T.

2. The method of claim 1, wherein the NTG-N(7-8)-WGATAR sequence, NAG-N(7-8)-WGATAR sequence or the reverse complementary sequence thereof is produced by one or more means selected from a group consisting of deletion, insertion, mutation and any combination thereof.

3-5. (canceled)

6. A ssODN comprising an NTG-N(7-8)-WGATAR sequence, NAG-N(7-8)-WGATAR sequence or reverse complementary sequence thereof;
wherein the reverse complementary sequence of WGATAR is YTATCW, wherein W is T or A, R is A or G, Y is T or C, and N is A, G, C or T.

7. The ssODN of claim 6, wherein the ssODN further comprises a 5′ homology arm and a 3′ homology arm;
preferably, the length of the 5′ homology arm and the 3′ homology arm are 20 to 300 nt;
and/or, the 5′ homology arm or the 3′ homology arm of the ssODN is selected from the sense strand or the antisense strand of the intended-editing region;
and/or, the 3′ terminus of the 5′ homology arm is 0-20 bases apart from the 5′ terminus of the 3′ homology arm in the ssODN.

8-9. (canceled)

10. The ssODN of claim 6, wherein the ssODN further comprises a substitution sequence forming the NTG-N(7-8)-WGATAR sequence, NAG-N(7-8)-WGATAR sequence or reverse complementary sequence thereof with bases upstream and/or downstream of the substitution sequence; wherein the base number of the substitution sequence in the ssODN is 0-6;
preferably, the ssODN comprises one or more sequence selected from the group consisting of SEQ ID NO: 4 to SEQ ID NO: 7, SEQ ID NO: 9 to SEQ ID NO: 22, SEQ ID NO: 24, SEQ ID NO: 26, SEQ ID NO: 28 to SEQ ID NO: 31, and the reverse complementary sequence thereof;
more preferably, the ssODN comprises one or more sequence selected from the group consisting of SEQ ID NO: 40 to SEQ ID NO: 65, and the reverse complementary sequence thereof;
for example, the ssODN comprises one or more sequence selected from the group consisting of SEQ ID NO: 45, SEQ ID NO: 52, SEQ ID NO: 53, SEQ ID NO: 54, SEQ ID NO: 56 and the reverse complementary sequence thereof; or, the ssODN comprises one or more sequence selected from the group consisting of SEQ ID NO: 52, SEQ ID NO: 53, SEQ ID NO: 54, SEQ ID NO: 56 and the reverse complementary sequence thereof.

11-13. (canceled)

14. The ssODN of claim 6, wherein the ssODN is chemically modified a ssODN with chemical modification; preferably, the chemical modification is phosphorothioate modification at the 5′ and/or 3′ terminus.

15-19. (canceled)

20. A pharmaceutical composition comprising the ssODN of claim 6, and a gene editing system targeting the promoter region of HBG1 or HBG2;
optionally, the gene editing system is used for introducing the NTG-N(7-8)-WGATAR sequence, NAG-N(7-8)-WGATAR sequence or reverse complementary sequence thereof with the ssODN.

21. The pharmaceutical composition of claim 20, wherein the gene editing system is a CRISPR-Cas editing system, a TALEN editing system, or a ZFN editing system;
preferably, the CRISPR-Cas editing system is a CRISPR-Cas9 system, or a CRISPR-Cas12 system;
more peferably, the Cas9 nuclease of the CRISPR-Cas9 system is derived from Streptococcus pyogenes.

22. (canceled)

23. The pharmaceutical composition of claim 21, wherein the CRISPR-Cas editing system comprises a guide RNA and Cas protein, or mRNA encoding Cas protein and gRNA, or a plasmid expressing Cas protein and gRNA;
preferably, the CRISPR-Cas editing system comprises a ribonucleoprotein complex composed of a guide RNA and Cas protein.

24-26. (canceled)

27. A kit for activating γ-globin gene, comprising:
(1) the ssODN of claim 6; and
(2) Cas9 or Cas12 protein, the DNA sequence encoding the same, the transcript thereof and/or recombination expression vector expressing the same.

28-29. (canceled)

30. The method of claim 1, wherein the enhancer element comprises the NTG-N(7-8)-WGATAR sequence or reverse complementary sequence thereof.

31. The method of claim 30, wherein the promoter region of γ-globin gene is selected from the group consisting of position −428 to position −66 of HBG1 gene promoter region, position −432 to position −66 of HBG2 gene promoter region, and any combination thereof.

32. The method of claim 30, wherein the promoter region of γ-globin gene is selected from the group consisting of position −92 to position −66 of HBG1 gene promoter, position −92 to position −66 of HBG2 gene promoter, position −129 to position −98 of HBG1 gene promoter, position −129 to position −98 of HBG2 gene promoter, position −175 to position −153 of HBG1 gene promoter, position −175 to position −153 of HBG2 gene promoter, position −192 to position −160 of HBG1 gene promoter, position −192 to position −160 of HBG2 gene promoter, position −428 to position −406 of HBG1 gene promotoer, position −432 to position −410 of HBG2 gene promoter, and any combination thereof.

33. The method of claim 1, wherein the promoter region of γ-globin gene is selected from the group consisting of position −129 to position −98 of HBG1 gene promoter, position −129 to position −98 of HBG2 gene promoter and any combination thereof.

34. The pharmaceutical composition of claim 23, wherein the DNA sequence corresponding to the guide sequence of gRNA is partially or completely the same with the gRNA recognition site sequence selected from the group consisting of SEQ ID NO: 32 to SEQ ID NO: 39; preferably, the gRNA is selected from the group consisting of SEQ ID NO: 69 to SEQ ID NO: 76, and any combination thereof.

35. A recombinant cell, wherein the γ-globin gene promoter region comprises an enhancer element containing NTG-N(7-8)-WGATAR sequence, NAG-N(7-8)-WGATAR sequence or reverse complementary sequence thereof;
wherein the reverse complementary sequence of WGATAR is YTATCW, wherein W is T or A, R is A or G, Y is T or C, and N is A, G, C or T.

36. A method for preparing the recombinant cell of claim 35, wherein the method comprises a step of administering the ssODN of claim 6.

37. The method of claim 36, wherein the target cell is a cell having differentiation potential into erythrocyte;
preferably, the cell is a stem cell or a progenitor cell having the differentiation potential into erythrocyte;
more preferably, the target cell is a hematopoietic stem cell.

38. A method for treating anemia in a subject in need of comprising administering an effective amount of the ssODN of claim 6 to the subject;
preferably, the anemia is β-Thalassemia or sickle anemia.

39. A method for treating anemia in a subject in need of comprising administering an effective amount of the pharmaceutical composition of claim 20 to the subject;
preferably, the anemia is β-Thalassemia or sickle anemia.
